(19) United States
(12) Patent Application Publication
(10) Pub. No.: US 2004/0013614 A1
(43) Pub. Date: Jan. 22, 2004

(54) Acrylic-based adhesive composition

(76) Inventors: Jose M Martin Martinez, Alicante (ES); Maria del Mar Mahiques Bujanda, Alicante (ES); Maria E Mulet Homs, Alicante (ES); Jorge L. Alio Y Sanz, Alicante (ES)

Correspondence Address: NIXON & VANDERHYE, PC, 1100 N GLEBE ROAD, 8TH FLOOR, ARLINGTON, VA, 22201-4714, US

(21) Appl. No.: 10/343,610

(22) PCT Filed: Aug. 1, 2001
(86) PCT No.: PCT/IB01/01840

(30) Foreign Application Priority Data
Aug. 2, 2000 (ES) 20001975

(51) Int. Cl. A61K 9/14; A61L 9/04

(52) U.S. Cl. 424/46

(57) ABSTRACT

An Adhesive composition for us in surgery, especially ophthalmic surgery, comprises a mixture of a cyanoacrylate of general formula (I) and an acrylate compound of formula (II) where R4 is an alkylene group and Y is a cycloalkyl group or Y═OR where R═H or acryloyl. It is found that compositions of this type have a number of advantages over simple cyanoacrylate adhesives, including: (a) a longer cure time, allowing easier handling of the joint; (b) a more flexible cured product; (c) a tendency for the cured to come away from the tissue after a month or so thus facilitating healing; and (d) a substantially non-exothermic curing reaction, thereby avoiding thermal damage to the tissue.

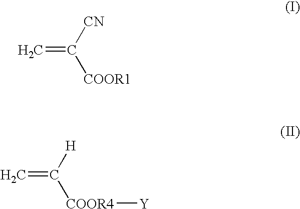

### INVENTION FIELD

The invention relates to bonding biological tissues and, in particular, relates to some acrylic-based adhesive compositions comprising various acrylate/cyanoacrylate mixtures. The invention also relates to procedures to prepare the adhesive compositions and to apply them in surgery.

### BACKGROUND OF THE INVENTION

Surgical suturing in general constitutes a secure method to fix biological tissues that have been dissected or have suffered some type of accidental trauma. The task of the suture in surgery is to seal the separated area and to facilitate the natural healing process. However, suturing involves additional trauma to the wound and sometimes it is unfeasible, for instance when a lot of biological tissue has been lost or when the tissue is weakened. Therefore, the search for alternatives to conventional sutures is currently a field of interest in surgery. Repeated attempts have been made to obtain histocompatible adhesives of broad efficacy in multiple fields of surgery.

In general, the advantages of adhesive bonding against suturing can be summarised as follows:

tissue bonding is achieved quickly;

forces tending to separate the edges of the wound or incision are distributed more evenly;

the additional trauma which suturing involves is avoided;

the risk of microbe contamination is reduced by sealing the wound or incision entirely;

using adhesives allows filling of intermediate areas of the wound that are difficult to access; and

surplus adhesive applied comes away from the healed wound.

The breakthroughs that have occurred in recent years are surprising, and virtually in all fields of surgery there are references to the use of different adhesives in occasional practices where traditionally sutures were used. Some key examples are as follows:

1. Cardiovascular surgery. Cyanoacrylate adhesives are used in forming vascular and microvascular unions. Some microvascular anastomoses have been performed using a polyethylene glycol seal on the site, and using a fibrin adhesive. This adhesive has also been used for sealing vascular grafts, implantation of vascular prostheses, in transposing major vessels and to stem bleeding during surgery.

2. Chest surgery. Fibrin adhesives have been used to seal oesophageal gastric anastomoses, to close tetraoesophageal and broncho-oesophageal fistulae, and, finally, to seal extra and intrapulmonary defects in parenchymal tissue when repeated surgical resections occur due to metastasis. Cyanoacrylic adhesives are used to control fluid draining through diaphragmatic surfaces and in broncho-pleural lesions.

3. Gynaecological surgery. Fibrin adhesives have been used to repair membranes that have broken prematurely during pregnancy and to control uterine bleeding.

4. Genital-urinary, plastic, neurological and ear nose and throat surgery. Fibrin adhesives have been used to seal anastomoses of the urethra, to hold grafts, to repair peripheral nerves and for timpanoplasties, respectively.

5. Orthopaedic surgery. Bone materials can be bonded using various adhesives: fibrin adhesives, cyanoacrylate adhesives, polyurethane adhesives, epoxy resins, gelatine-based adhesives and marine-protein adhesives.

In ophthalmic surgery adhesives have been used, but are only used in a very limited way owing to problems relating to tolerance and handling, in the following procedures:

sealing traumatic perforations and corneal ulcers;

union of artificial membranes to front and back surface of the eye chamber (artificial epithelium and endothelium);

adhesives in prostheses that penetrate the cornea;

sealing conjunctive perforations; and

performance of scleral loops without suture.

Fibrin adhesives dissolve quickly and are absorbed in vivo, and they have an adequate biological tolerance. Nevertheless, there are biological-origin products liable to suffer viral contamination. Moreover fibrin shows poor cohesive properties and results in very weak adhesive unions.

Cyanoacrylic adhesives are single-component synthetic adhesives that polymerise rapidly in the presence of water. Among the most used cyanoacrylate adhesives in surgery are butyl, isobutyl and octyl derivatives. These adhesives show extraordinary adhesion to biological tissue in a few seconds. However, cyanoacrylate adhesives present serious drawbacks when applying them to biological substrates, such as:

polymerisation is an exothermic reaction that can lead to localised high temperatures when the aqueous content of the tissue is significant;

the reticulated polymer is very rigid, which causes ulceration of surrounding tissue; and

the reticulated polymer requires at least two months to release itself from the tissue to which it has been applied, thus delaying the natural healing process.

Therefore use of cyanoacrylate adhesives in ophthalmology is restricted to extreme situations where an immediate seal is required before proceeding with repair surgery.

Mixtures of carboxyl and cyanoacrylic adhesives constitute a family of adhesive compositions developed by the research team applying for this patent. From the chemical viewpoint, they involve mixtures of carboxyacrylate and an alkyl cyanoacrylate in a ratio that can range from (1:1) to (3:7) by volume. The carboxyacrylic derivative acts as adhesion modulator and, in particular, reduces the rigidity of the cyanoacrylates, and provides a greater capacity to wet the tissue. These compositions have adequate reticulation time, produce very strong adhesive unions, have good tissue tolerance and, moreover, the time to eliminate the polymerised product is less than one month and does not interfere with healing. These adhesive mixtures are suitable for lazy-eye operations in ophthalmic surgery, but in conjunctive replacement they do not respond adequately because the reticulated adhesive is too rigid. In addition, a twin-component mixture is involved that needs to be prepared during clinical application, which adds another disadvantage.

### SUMMARY OF THE INVENTION

The invention addresses these problems by providing a new species of histocompatible adhesives suitable for joining biological tissue.

The invention is based in the discovery that various hydroxy and cycloalkyl acrylates improve the performance of the alkyl cyanoacrylates usually used in histocompatible adhesive compositions. Thus the invention provides a histocompatible adhesive composition comprising an acrylate and cyanoacrylates in accordance with claim 1. These compositions can replace traditional suturing of biological tissue in surgery, as shown by experiments to join conjunctive surfaces to the ocular sclera, which show excellent biological tolerance (see Example 3).

An additional aspect of this invention is a procedure to obtain this adhesive composition.

A further aspect of this invention is the use of this adhesive composition in surgery.

Another aspect is the use of a composition according to claims 1 to 10 to produce an histocompatible adhesive composition for joining tissue in surgery.

The invention provides an acrylic-based histocompatible adhesive composition, hereafter adhesive composition of the invention, comprising:

(a) at least one cyanoacrylate (1) of general formula

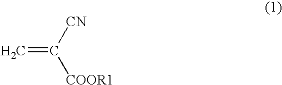

where R1 is an alkyl group or a —R3-OR2 group, where R2 and R3 are alkyl and alkylene groups respectively; and

(b) at least one acrylate (2) having the general formula

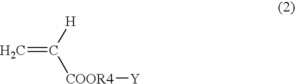

where R4 is an alkylene group and Y is a cycloalkyl group or Y═OR where R═H or acryloyl.

Preferably R1 is a C₁-C₃₀ alkyl group or a —R3-OR2 group wherein R2 is a C₁-C₃₀ alkyl group.

More preferably, R1 and R2 are C₁-C₁₀ alkyl groups.

R4 is preferably a C₂-C₈ alkylene group, more preferably C₄-C₇ alkylene, and most desirably is C₆H₁₂.

Y is preferably a terminal —OH group. R may be acrylate in which case R4 is C₂H₄.

The volume ratio of compound (1) to compound (2) is preferably between 3:7 and 7:3 inclusive.

### DETAILED DESCRIPTION OF THE INVENTION

The following acrylate compounds were prepared and mixtures of these compounds with n-butyl cyanoacrylate were prepared in various volume ratios.

A.1 4-Hydroxybutyl acrylate

A.2 1-Hydroxybutyl acrylate

A.3 5-Hydroxypentyl acrylate

A.4 6-Hydroxyhexyl acrylate

A.5 2-Cyclohexylethyl acrylate

A.6 2 Hydroxy-5-methoxyphenyl acrylate

A.7 Ethylene glycol diacrylate

A.8 4-Hydroxybutyl diacrylate

A.9 5-Hydroxypentyl diacrylate

A.10 Polyethylene glycol diacrylate

A.11 Propylene glycol acrylate.

These compounds are referred to generally as compound (2) in the mixtures, whilst the cyanoacrylate component of the mixtures is referred to as compound (1).

General formula cyanoacrylates (1) are known compounds that can be obtained by conventional methods, i.e. by esterification of cyanoacrylic acid with the corresponding alcohol. In the invention's adhesive composition there can be one or more general formula cyanoacrylates (1) present.

The term “C₁-C₃₀ alkyl group” as used in this description includes any radical derived from a linear or ramified alkane with 1 to 30 carbon atoms. In a specific formulation, the alkyl group present in the cyanoacrylate (1) is an ethyl or butyl radical, e.g. n-butyl, sec-butyl or tert-butyl.

In another particular formulation, the cyanoacrylate (1) is a compound where R1 is a —(CH₂)n-O—R2, where a selection is made between 1 and 2 and where R2 is ethyl, i.e. R1 is ethoxymethyl [—CH₂—O—CH₂—CH₃] or ethoxyethyl [—CH₂—CH₂—OCH₂—CH₃], respectively.

Specific examples of formula cyanoacrylates (1) include: ethyl cyanoacrylate, n-butyl cyanoacrylate, sec-butyl cyanoacrylate, tert-butyl cyanoacrylate, ethoxymethyl cyanoacrylate and methoxyethyl cyanoacrylate.

The (1):(2) ratio present in the adhesive composition of the invention can vary within a very broad interval, depending on the application for which the invention's adhesive composition is used, preferably in a ratio, by volume, of X:(10−X) , where X represents the parts in volume of (2) and is between 3 and 7, both inclusive and (10−X) represents the parts in volume of cyanoacrylate (1).

Synthesis Procedure

1. Synthesis of Acrylic Acid

50 g acrylonitrile and 90 ml HCl conc. were added in a three-necked round flask. The mixture was maintained under reflux for three hours. The reaction product was submitted to acid-base extraction.

2. Synthesis of Acrylic Derivatives

The acrylic acid and the alcohol used in the synthesis of the acrylic derivatives were reacted under reflux using a minimal amount (about 0.5 ml) of sulphuric acid as catalyst of the reaction. After 3 hours, the reaction product was diluted in ethyl acetate and washed till neutral in the aqueous phase. Finally, the product was purified in a chromatographic plaque.

3. Structural Elucidation of the Acrylic Derivatives

The following spectroscopic techniques were used:

IR Spectroscopy

¹H Nuclear Magnetic Resonance

¹³C Nuclear Magnetic Resonance

4. Prepation of the Adhesive Mixtures

Acrylic and cyanoacrylic derivatives were mixed in different amounts (v/v) (3:7)(4:6)(1:1)(6:4).

The stability of the mixtures was monitored using IR spectroscopy. This technique shows evidence of the olefin groups (1643 cm⁻¹) involved in the polymerisation reaction.

The stability of all mixtures was monitored for 3 months. The mixtures were kept at −3° C. under nitrogen atmosphere.

The formulations chosen were mixtures with n-butyl cyanoacrylate as follows, with the ratio of (2):(1) shown in brackets:

A.1 (4:6)

A.2 (4:6)

A.3 (4:6)

A.4 (4:6)

A.5 (3:7)

A.7 (3:7)

In Vivo Tests

Substrate: Eyes of New Zealand white rabbit.

Surgery Practice

Anaesthesia medication included Ketamine (67 mg/kg) and Rompun (8 mg/kg). The animal was anaesthetised, a palpebral incision was produced using a “blefarostatus”. An 8 mm peritoneal incision was made at the superior limbous tissue. A resection of the tenon was carried out.

Subconjunctival tissue was cut at the two ends where 2 drops of the freshly prepared adhesive mixture were placed. Conjunctival tissue was put back into place and slight pressure applied for 30 seconds. After the surgical procedure, some antibiotic drops were placed in each eye.

Protocol: Animals were treated following the protocol establish in the ARVO recommendations.

20 eyes of 20 adult rabbits were used for each adhesive mixture tested. A rabbit group using a traditional suture procedure was used as control.

The macroscopic observation of the rabbit eyes included the following aspects:

Evaluation of conjunctiva injection.

Presence of secretion.

Residues of adhesive.

Hardness of adhesive residues.

These parameters were classified as: absence, light, moderate, severe. In each group, ten animals were sacrificed one week after the surgical procedure; the remaining rabbits were kept alive for 1 month.

The microscopic control included an histological analysis. Tissue inflammation grade of acute phase and chronic phase was evaluated.

### III-Experimental Results

A.1 Mixture (4-hydroxybutyl)

This mixture was a liquid with low viscosity. The mixture needed conjunctival infiltration during the surgery practice to be effective. Adhesion was completed in few seconds, and the aspect of the cured adhesive was slightly brittle, but acceptable.

A.2 Mixture (1-hydroxybutyl)

Formulations containing 1-hydroxybutyl acrylate seemed to be less reactive than those containing 4-hydroxybutyl acrylate (A.1 Mixture). Viscosity was too high and this caused the rejection of the mixture during the macroscopic evaluation.

A.3 Mixture (5-hydroxypentyl)

Viscosity of mixtures containing 5-hydroxypentyl acrylate was suitable. Adhesion was produced in one minute. The excess of the cured product was not rigid. The live animals had no secretions, no conjunctiva injection, and adhesive was totally degraded (i.e. disappears) in one month. Histological tests showed a degree of necrotic reaction of the biological tissue, but the result was generally acceptable.

A.4 Mixture (6-hydroxyhexyl)

Because of its excellent adhesion and chemical properties, the 6-hydroxyhexyl acrylate mixture provided the best performance compared to the other mixtures. Macroscopic evaluation of the animals suggested its biological tolerance, which was later confirmed by the histopathological results.

A.5 Mixture (2-cyclohexylethyl)

Adhesive formulation containing 2-cyclohexylethyl acrylate had limitations when used “in vivo”, because the time necessary for joining the incision was slightly too long (about 3 minutes). The joint strength was not as good as that obtained with the other derivatives but its biological tolerance was good. This mixture was therefore acceptable, though with a less than ideal curing time.

A.7 Mixture (Ethylene Glycol Diacrylate)

Probably because of its molecular size, the ethylene glycol diacrylate mixture showed the same limitations as the A.5 Mixture. Its behaviour in in vivo assays was not entirely successful. Histological tests rendered evidence of a slight inflammatory process. The result for this mixture is therefore just acceptable, but with quite serious limitations.

The A.4 mixture was generally superior to the rest, so further experimental work was conducted with this mixture. However, it is to be expected that similar testing to that conducted on the A.4 mixture would produce analogous results with the A.1-A.3, A.5 and A.7 mixtures.

In Examples 2 and 5 we describe the preparation of several adhesive compositions that comprise A.4 and a cyanoacrylate (1) in different volumetric ratios [3:7, 4:6 and 5:5].

The invention's adhesive composition can be obtained by means of a process involving the mixture of the various components in suitable ratios, depending on the application in which the invention's adhesive composition is to be used. The result of mixing the components constitutes a single stage. This mixture is stable (see Example 2) under suitable storage conditions, where keeping of the invention's adhesive composition is stored in containers, preferably opaque, such as topaz, which are vacuum packed and stored at a temperature of 5° C. to 15° C.

The (co) polymerisation of monomers after applying the invention's adhesive composition to the biological tissues to be joined is triggered by action of the water present in the substrates and provides an histocompatible adhesive suitable to join biological tissues.

Because of its mechanical, viscoelastic and biotolerance properties, equal to or greater than other adhesive of biological or synthetic origin described hitherto, the invention's adhesive composition can be used as tissue adhesive in surgery in general, and in ophthalmic surgery in particular, for example as conjunctive sealant, where it can replace suture where the conjunctiva joins the sclera. In vivo experimentation with adhesive compositions provided by this invention has been conclusive regarding its good histological tolerance, both macroscopic and microscopic. Gradual degradation of the invention's adhesive composition once applied and the subsequent gradual coming away from both tissues also helps the healing process.

Example 3 shows the efficacy of an adhesive composition comprising A.4 and n-butyl cyanoacrylate in a volumetric ratio of 4:6 for in vivo bonding of the conjunctiva to the sclera in the limbar zone.

Therefore, along other lines, the invention refers to the use of a mixture comprising 6-hydroxyhexyl acrylate and at least one general formula cyanoacrylate (1) previously shown in the preparation of an acrylic-based adhesive composition, histocompatible, to bond biological tissues in surgery, specifically to join the conjunctiva to the sclera in ophthalmic surgery.

Adhesives obtained from the adhesive compositions provided by this invention have physico-chemical and biological properties more suitable for use in conjunctive replacement than commercial bioadhesives that can be used currently and they have, among others, the following characteristics:

ease of handling as they are stable mixtures for a relatively long time;

good wettability of biological substrates;

tissue sealing time is less than 5 minutes, preferably less than 1 minute from application;

excellent macroscopic and microscopic tolerance;

suitable flexibility of the polymerised product; and

elimination of polymerised product applied to tissue joins takes no longer than 1 month from application.

Moreover, the invention's adhesive composition has several advantages both over known bioadhesives used in surgery and over traditional suture.

Alternative use of an adhesive composition provided by this invention instead of known bioadhesives used in surgery [fibrin and n-butyl cyanoacrylate adhesives] assayed in operations provides, among others, the following advantages:

a) sterilisation of the invention's adhesive composition can be controlled from preparation time;

b) easier handling as the invention's adhesive composition is a chemically stable mixture;

c) polymerisation of the mixture is fast, but not instant (some 45 seconds, at least for A.4) and occurs with a small heat production; moreover as there is some time before reticulation of the adhesive composition occurs, any excess of the applied adhesive composition can be removed;

d) the adhesive capacity of the invention's adhesive composition makes it possible to keep the tissue substrates joined firmly throughout the healing process;

e) reticulated adhesives are transparent and are not too rigid, thus the tissues surrounding the adhesive join do not suffer from ulcer-type lesions;

f) the invention's adhesive composition has excellent histological tolerance, both macroscopic and microscopic;

g) biodegradation of the polymerised adhesive occurs in less than one month, in line with the natural healing time; and

h) its financial cost is lower.

The adhesive compositions provided by this invention are effective in joining biological substrates and avoid the problems arising in surgical use of traditional suture. E.g. in joining the conjunctiva to the sclera in ophthalmic surgery, the advantages of the invention's adhesive compositions over traditional suture are summarised as follows:

a) time saved during operation;

b) they enable highly damaged histological fractions that could not be sutured to be respected;

c) additional trauma and histological irritation from suture in biological tissues surrounding the replaced tissue is avoided; and

d) appearance of the tissue after applying the adhesive mixture is clean, even and non-traumatic.

Dosage of the adhesive composition provided by this invention will be established by the consultant on the basis of several factors, such as tissues, the surface to be joined, state of the wound, etc. In a specific case, the invention's composition can be applied topically in a quantity of approximately 0.2 ml ml/cm² of tissue surface.

The following examples serve to illustrate the invention and should not be considered as being restrictive on its scope.

### EXAMPLE 1

### 6-hydroxyhexyl Acrylate Synthesis

1.1 Acrylic Acid Synthesis

In a ground reaction volumetric flask add 50 g of acrylonitrile (0.94 mol); connect the system to a backflow refrigerant and start heating and mechanical shaking. When the temperature reached at the heart of the volumetric flask is approximately 50° C., add 90 ml of concentrated hydrochloric acid. Leave the mixture to react at 75° C. for 50 minutes. Then extract the acrylic acid from the reaction mixture by means of acid-based fractioning. Transfer the mixture to a decanting funnel, remove the aqueous phase containing mainly ammonium chloride and excess hydrochloric acid. Add the organic phase slowly on an Erlenmeyer where beforehand 24 g of sodium hydroxide have been dissolved in 83 ml of distilled water; this operation should be carried out over an ice bath in order to dissipate any heat given off by this exothermal acid-based reaction. After adding, take the contents of the volumetric flask to a decanting funnel to separate the organic phase that has not reacted in the base medium. Acidify the aqueous phase with 10% diluted sulphuric acid, add 50 ml of diethyl ether and extract the organic phase. Rinse it, dry it on anhydrous sodium sulphate, filter it and distil it to provide 20 g of a liquid product at room temperature, which is transparent and has a low boiling point. Analyse it by infrared spectroscopy and proton nuclear magnetic resonance.

1.2 6-hydroxyhexyl Acrylate Synthesis

React 20 g (0.27 mol) of the acrylic acid obtained before using Example 1.1 with an equimolar quantity of the 1,6-hexanediol [38.2 g (0.27 mol)] at 90° C. for 3.5 hours using electromagnetic shaking, with backflow and with a small quantity of sulphuric acid present as reaction catalyst.

After this time, transfer the reaction mixture to a decanting funnel and add 50 ml of ethyl acetate and 100 ml of distilled water; extract the aqueous phase and discard. Wash the organic phase up to neutral pH of the rinsing waters. Dry the organic phase on anhydrous sodium sulphate, filter it and distil it to provide 35 g (0.25 mol) of a liquid product at room temperature that is slightly yellowish, whose chemical structure is corroborated by infrared spectroscopy, proton nuclear magnetic resonance and carbon-13 nuclear magnetic resonance. The yield from this reaction is in the order of 60%.

The resulting infrared spectrum enables elucidation of the major functional groups of its structure. The more significant bands and allocations to the relevant functional groups are as follows:

| Functional group | Wave number (cm⁻²) |
|---|---|
| Alcohol | 1401, 3543 |
| Ester | 1246, 1743 |
| Exocyclic methylene | 897, 1643 |

The proton nuclear magnetic resonance spectrum evidences the protons present in the molecule, will the following signals being picked up: 3 groups of double doublet signals (8.2, 7.6 and 7.1 ppm, Jab: 17 Hz, Jbc: 2 Hz and Jac: 10 Hz), that can be allocated to an abc system consisting of the three olefin protons of the molecule; 1 triple strength triplet signal at 4.16 ppm, attributable to protons on the rest of the ester; one double signal at 3.64 ppm for protons on the carbon supporting the hydroxyl group; and a multiplet signal at 1.46 ppm, that can be allocated to 8 protons of the alkyl remainder.

The carbon-13 nuclear magnetic resonance spectrum gives 9 signals, 7 of which that can be allocated to methylene carbons, of which one is olefm and another two support an oxygenated function (139.1, 68.5, 63.0, 33.1, 26.3, 29.2, 32.4 ppm); 1 signal attributable to a quaternary carbon (174 ppm) and, finally, 1 signal for a methyl carbon (136.6 ppm).

All this data is in line with the proposed structure of 6-hydroxyhexyl acrylate.

### EXAMPLE 2

### Chemical Stability Assay

One of the main advantages of the new adhesive compositions provided by this invention is the chemical compatibility between the two acrylic components, which allows it to be bottled in a single container, with no additives to inhibit the formation of free radicals or anionic polymerisation.

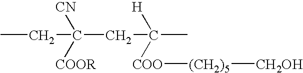

The chemical stability of several monomer mixtures [A.4 and cyanoacrylate (1)], specifically (i) A.4 and n-butyl cyanoacrylate, with a ratio of A.4:butyl cyanoacrylate of 4:6 in volume, (ii) A.4 and isobutyl cyanoacrylate, with a ratio of A.4:isobutyl cyanbacrylate of 4:6 in volume and (iii) A.4 and ethyl cyanoacrylate, with a ratio of A.4:ethyl cyanoacrylate of 4:6 in volume, it was shown by regular assays by means of proton magnetic resonance, of aliquots taken at time intervals from 0 minutes to 2 weeks from preparation. The various mixtures were prepared by mixing A.4 and the relevant cyanoacrylate (1) in the desired volumetric ratio. They were vacuum-bottled in topaz jars and stored at temperatures ranging from 50° C. to 150° C.

The proton nuclear magnetic resonance spectra did not evidence signs of doublet or singlet multiplicity attributable to protons in the carbonylic carbon β present in the fully or partly polymerised product.

### EXAMPLE 3

### In Vivo Bonding of the Conjunctiva to the Sclera in the Zone

For this assay we used an adhesive composition provided by this invention comprising 6-hydroxyhexyl acrylate and n-butyl cyanoacrylate in a 4:6 ratio on volume. For controls we used animals that had been operated on and treated with suture.

3.1 Surgical Operation Protocol

In an anaesthetised rabbit, separate a fragment of the conjunctiva from the limbar edge, running 180° relative to the cornea. Clean and dry the scleral tissue under the retracted conjunctiva using a haemostat and instil the adhesive composition forming a constant line along the limbar edge. Retake the conjunctiva and put it back in its original position, pressing lightly on the adhered area for a few seconds. Wait for 2 minutes, and we can see that the tissue join is now firm, completing the operation.

3.2 Results

Assessment of results is done by operating on two animal groups in parallel following the surgical procedure described in Example 3.1 and using suture or the adhesive composition.

The acute post-surgical inflammatory reaction evidenced after 24 hours is considerably less in the group operated on and treated with the adhesive composition than the control group (treated with surgical suture); the same occurs with hyperaemia and ocular tears.

The animals are put to sleep after one month. We verified that there was no adhesive left and that the tissue join was firm and even. There was no evidence of any granulomatosis reactions associated with chronic-type inflammatory processes.

### EXAMPLE 4

### Assessment of the Strength of the Joins

To assess the strength of adhesive joins generated by the invention's adhesive compositions, we carried out a protocol for adhesion to reproducible inert surfaces.

For the specific performance of this assay we used an adhesive composition provided by this invention comprising 6-hydroxyhexyl acrylate and butyl cyanoacrylate in a ratio of 4:6 in volume and an adhesive composition comprising ethyl cyanoacrylate as control.

4.1 Protocol

Take 20 tubes made of thermoplastic rubber (TR) 300 mm wide, 200 mm long and 3 mm thick. Clean the surface to which the adhesive composition is to be applied using MEK (methyl ethyl acetone). Wait 15 minutes and apply a commercial initiator for polyolefins on a 3×3 cm surface at one end of the tubes. After 15 minutes apply a thin coat of the adhesive (some 20 μm), either ethyl cyanoacrylate or an adhesive that represents this invention [A.4:butyl cyanoacrylate (4:13) in volume] to the treated surface and join them overlapping the ends of the tubes partially in pair.

4.2 Results

Separate the 10 TR rubber joins made with the various adhesives using a universal assaying machine 24 hours after bonding at a speed of 20 mm/min. Establish the energy and force per unit of length (×30 mm) needed to separate the two TW rubber tubes.

Joins made with ethyl cyanoacrylate give a separation resistance of 8.3 kN/m, obtaining cohesion failure in the rubber; for the invention's adhesive composition separation resistance was 5.5 kN/m , with cohesion failing in the adhesive. The reduction in strength of the adhesive join is due to reduction in the adhesive's cohesive strength. Nevertheless, this reduction in adhesive capacity of the new compositions is not relevant when joining biological substrates as the stresses to which these joins are subjected are never as demanding (before the adhesive join can separate there the tissues would tear); however, the flexibility and biological tolerance given by the invention's adhesive composition are better than the ethyl cyanoacrylate adhesive.

### EXAMPLE 5

### Preparation of Adhesive Compositions

The following adhesive compositions were prepared by mixing the various components in the desired volumetric ratio and stored in topaz flasks in a vacuum.

| Components | Ratio in volume A.4:(1) |
|---|---|
| A.4 + n-butyl cyanoacrylate | 4:6 |
| A.4 + n-butyl cyanoacrylate | 5:5 (1:1) |
| A.4 + n-butyl cyanoacrylate | 3:7 |
| A.4 + ethyl cyanoacrylate | 4:6 |
| A.4 + ethyl cyanoacrylate | 5:5 (1:1) |
| A.4 + ethyl cyanoacrylate | 3:7 |
| A.4 + isobutyl cyanoacrylate | 4:6 |
| A.4 + isobutyl cyanoacrylate | 5:5 (1:1) |
| A.4 + isobutyl cyanoacrylate | 3:7 |

1. An adhesive composition comprising:
(a) at least one cyanoacrylate (1) of general formula 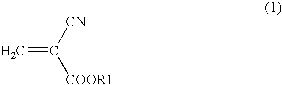
where R1 is an alkyl group or a —R3-OR2 group, where R2 and R3 are alkyl and alkylene groups respectively; and
(b) at least one acrylate (2) having the general formula 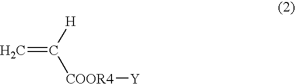
where R4 is an alkylene group and Y is a cycloalkyl group or Y═OR where R═H or acryloyl:

2. An adhesive composition as claimed in claim 1 wherein R1 is a C₁-C₃₀ alkyl group or a —R3-OR2 group, and wherein R2 is a C₁-C₃₀ alkyl group.

3. An adhesive composition as claimed in claim 2 wherein R1 is a C₁-C₁₀ alkyl group or a —R3-OR2 group, and wherein R2 is a C₁-C₁₀ alkyl group.

4. An adhesive composition as claimed in claim 1 wherein compound (1) is chosen from ethyl cyanoacrylate, n-butyl cyanoacrylate, sec-butyl cyanoacrylate, tert-butyl cyanoacrylate, ethoxymethyl cyanoacrylate, methoxyethyl cyanoacrylate and mixtures thereof.

5. An adhesive composition as claimed in any of claims 1 to 4 wherein R4 is a C₂-C₈ alkylene group.

6. An adhesive composition as claimed in claim 5 wherein Y is a terminal —OH group.

7. An adhesive composition as claimed in claim 6 wherein R4 is a C₄-C₇ alkylene group.

8. An adhesive composition as claimed in claim 7 wherein R4 is C₆H₁₂.

9. An adhesive composition as claimed in any of claims 1 and 5 wherein R is acrylate and R4 is C₂ H₄.

10. An adhesive composition as claimed in any preceding claim wherein the volume ratio of compound (1) to compound (2) is between 3:7 and 7:3 inclusive.

11. An adhesive composition as claimed in claim 1 comprising 6-hydroxyhexyl acrylate and n-butyl cyanoacrylate in a volume ratio of 4:6.

12. Use of a composition according to any of the preceding claims in the production of a histocompatible adhesive composition to join biological tissue in surgery.

13. Use of a composition according to any of claims 1 to 11 in the production of a histocompatible adhesive composition to join the conjunctiva to the sclera in ophthalmic surgery.
